(19) United States
(12) Patent Application Publication
(10) Pub. No.: US 2007/0014819 A1
(43) Pub. Date: Jan. 18, 2007

(54) Method of emulsifying phytosterol by natural saponin,emulsion prepared thereby and water dispersible phytosterol powder product

(75) Inventors: Wen-Teng Wu, Tainan (TW); Jech-Wei Chen, Hsinchu (TW); Hsin-Ju Hsieh, Hsinchu (TW)

Correspondence Address: PATENTTM.US, P. O. BOX 82788, PORTLAND, OR, 97282-0788, US

(73) Assignee: WECKTECK BIOTECHNOLOGY COMPANY LIMITED, Taipei (TW)

(21) Appl. No.: 11/179,472

(22) Filed: Jul. 13, 2005

(51) Int. Cl. A61K 31/704; A61K 31/56

(52) U.S. Cl. 424/400; 514/26; 514/33; 514/171

(57) ABSTRACT

Method of emulsifying phytosterol by natural saponin is disclosed including mixing an oil phase having phytosterol dissolved therein and an aqueous phase having a natural hydrophilic surfactant (saponin) therein to obtain an emulsion of phytosterol. A water dispersible phytosterol powder product can be formed by drying the emulsion, which is able to be dispersed in an aqueous solution or beverage. The phytosterol emulsion, the phytosterol powder product and the aqueous dispersion thereof may be used in the cosmetic products and foodstuffs, thereby enhancing the value of the phytosterol.

### FIELD OF THE INVENTION

The present invention relates to a method of emulsifying phytosterol, particularly a method of emulsifying phytosterol by natural saponin.

### BACKGROUND OF THE INVENTION

Phytol has a full name of phytosterol and includes three major categories: C27 4-desmethyl, C28 4-desmethyl, and C29 4-desmethyl, for examples desmosterol, campesterol, dihydrobrassicasterol, sitosterol, stigmasterol, etc. Phytol and cholesterol have similar molecular structures. Therefore, phytol will interfere human intestine's absorption of cholesterol, and can be used to prevent the occurrence of excessive intake of cholesterol, thereby reducing the risk of cardiovascular diseases. Furthermore, phytol, based on previous literature disclosures, has important physiological activities, including enhancing human immunity and reducing risk of cancer.

According to the statistics in Taiwan last year, many of the top ten causes of death (including cerebrovascular diseases, heart diseases, high blood pressure, etc.) are related to the high concentration of cholesterol in blood. In comparison with the situation in the past, the percentages of patients with these diseases are on the rise steadily year-after-year. It has become an important medical issue on how to reduce the high concentration of cholesterol in blood.

At present, commercial medicines for reducing cholesterol in blood mainly are Satins, which are suitable for patients suffering from primary hypercholesterolemia and can be used to effectively reduce LDL cholesterol, as well as slightly reducing triglyceride, and increasing HDL cholesterol. Statins have severe side effects including Rhabdomyolysis. For example, the medicine Baycol (cerivastatin), developed and marketed by Bayer Co. in the U.S.A. in 1997, has caused more than 100 people around the world dead from the side effects of this medicine. As a result, Bayer Co. is facing lawsuits from around the world demanding huge amounts of compensation.

Presently, the best selling Statins medicine is Lipitor from Phizer Co. Lipitor has an effect of reducing about 29%˜45% of total cholesterol according to different prescriptions.

The effect of phytosterol in reducing cholesterol in blood has been disclosed in dozens of medical articles. Generally speaking, daily intake of 1.4 g of phytosterol for one month can reduce about 14% of total cholesterol in blood. Even though in comparison with Statins, phytosterol is less effective in reducing total cholesterol in blood, phytosterol has the following advantages: natural, no side effects, and low production cost, etc. Thus, FDA of the U.S.A. published an article (TOO-40) on ₅th of September, 2000 recognizing that a daily intake of more than 1.3 g of phytosterol can effectively reduce the concentration of LDL, thereby effectively reducing the risk of cardiovascular diseases (e.g. coronary heart disease).

Studies on the use of phytosterol in reducing cholesterol in blood started about 50 years ago. But, the first commercial phytosterol product was marketed by Raisio Co. in Finland in 1995. The dominant factor for such a slow progress in commercialization of phytosterol lies in that purified phytosterol is difficult to be dissolved in water or oil and most solvents. This is why a phytosterol product is difficult to be developed. Furthermore, when administered orally directly, purified phytosterol has an absorption rate of less than 5% in a human body, while cholesterol has an absorption rate of more than 50% in a human body. For the sake of product formulation and increasing the use rate of phytosterol, phytosterol needs to be converted into the form of a suitable food additive through some processing or formula conversion. For example, Raisio of Finland and the Unilever Co. (which subsequently marketed a similar product) used an unsaturated aliphatic acid to esterify phytosterol. Such an esterified phytosterol has a good oil solubility and can be added into butter or an oil product. Esterified phytosterol is a first derivative and is also a most prevalent derivative form (WO 9956558, in 1999; U.S. 2002/0160990, in 2002; U.S. Pat No. 6,492,538, in 2002). However, the biggest disadvantage of an oil soluble formula lies in that a large amount of grease will unavoidably be taken concurrently with the intake of phytosterol, which might cause other problems such as obesity.

A typical process of emulsifying phytosterol mainly includes wrapping phytosterol with an emulsifier to form liposome in an aqueous solution in order to greatly increasing the effects of phytosterol. In the miniaturization aspect, phytosterol particles are reduced in size as much as possible by using crystallization or dispersion techniques in order to enhance the effect of reducing the absorption of cholesterol of phytosterol. A phytosterol derivative which is the most difficult to be made is a water soluble derivative that can be added directly into soft drinks or water for achieving the highest absorption. This type of derivative needs to be made by a more complex chemical synthesis process to alter the chemical structure of phytosterol and requires more considerations on the issue of food safety. Forbes Medi-Tech Co. of the U.S.A. has successfully developed a water soluble phytosterol derivative by grafting Vitamin C onto phytosterol in order to increase the water solubility of the resulting phytosterol derivative. This water soluble phytosterol derivative is recognized as a new medicine and is under a phase II clinical trial in the U.S.A. Also, this water soluble phytosterol derivative is disclosed in U.S. Patent Publication Ser. No. 2002/0156051 A). Meanwhile, many patents have disclosed methods for emulsifying phytosterol into micelles in order to disperse and suspend phytosterol in water (U.S. Pat. No. 5,932,562, in 1999; U.S. Pat. No. 6,063,776, in 2000; WO 02/065859, in 2002; WO 02/100412, in 2002; JP 2002/291442, in 2002). Up to now, the emulsifier formulas used in the emulsification of phytosterol include lecithin or chemically synthesized surfactants. Even though these formulas can achieve the purpose of emulsification, they have inherent disadvantages, such as a higher cost associated with lecithin, a large amount of aliphatic acid required in a chemically synthesized surfactant. Therefore, the present invention intends to develop a better technique for the emulsification of phytosterol.

Saponin is a natural emulsifier extracted from plant. For example, Tea saponin is extracted from Camellia oleifera seeds; Sapindaceae is extracted from Sapindus seeds; Quillaia is from a water extract of smashed inner layer of bark or timber; and Yucca extract is extracted from a desert plant in the south western part of the U.S.A. Natural saponins can decompose grease and alter the permeability of cells to promote the absorption of medicine, and is capable of altering the surface tension of water. As a result, saponins can be used as an additive in food and soft drinks, as well as a promoter for forming natural foams in food. Many documents have mentioned the effects of saponins in reducing cholesterol level and saponins being safe as a food additive. Clinical trials indicate that Yucca saponin extract has a conspicuous effect in reducing human cholesterol level.

### SUMMARY OF THE INVENTION

In the present invention, many experiments have been carried out to test the emulsification stability of various saponins to phytosterol. The phytosterol emulsions are then dried by a frozen drying process or a spray drying process, etc. Subsequently, the dispersion properties of the dried powders in an aqueous solution were observed. The results indicate that Tea saponin, Sapindaceae, Yucca extract, and Quillaia extract are all capable to form stable emulsions and aqueous dispersions of phytosterol. On the overall consideration on food safety and re-dispersion property of an emulsified and dried powder in an aqueous solution, Tea saponin and Quillaia extract have the best performance.

A synergistic effect to the physiological activities of phytosterol can be achieved by forming an emulsified phytosterol mixture by using natural saponin. This type of emulsification formula is superior than other emulsification formulas achieved by using other artificially synthesized emulsifiers.

### DETAILED DESCRIPTION OF THE INVENTION

The present invention discloses a method for emulsifying phytosterol, which comprises the following steps:

a) dissolving phytosterol in an oil phase;

b) dissolving a saponin in a water phase; and

c) mixing said oil phase solution from step a) and said water phase solution from step b) to form an emulsion.

Preferably, the method of the present invention further comprises:

d) removing a major portion of said oil phase from said emulsion by evaporation under a reduced pressure; and

e) drying the resulting oil-in-water emulsion from step d) to obtain a phytosterol powder product containing said saponin and phytosterol, which is capable of being re-dispersed in a water phase.

Preferably, said oil phase in step a) comprises an organic solvent with a boiling point lower than water, and said step d) comprises distilling said emulsion from step c). More preferably, said oil phase in step a) comprises isopropanol.

Preferably, said oil phase in step a) comprises a vegetable oil.

Preferably, said oil phase in step a) comprises an animal oil.

Preferably, said saponin is a natural saponin extracted from a plant. More preferably, said plant is selected from the group consisting of Camellia Oleifera plant, Sapindus plant, Quillaia plant, and Yucca plant.

The present invention also provides a phytosterol emulsion, which is prepared according to the method of the present invention.

The present invention further provides a phytosterol powder product containing saponin and phytosterol, which is prepared according to the method of the present invention.

Other features and advantages of the present invention will be further elaborated through the following examples, which are for illustrative purpose and not for limiting the scope of the present invention.

### EXAMPLE 1

Table 1 lists the ingredients (in wt %) used in a phytosterol emulsion.

TABLE 1

| Ingredient | wt % |
|---|---|
| Phytosterol | 2.73 |
| Propylene glycol monostearate | 2.73 |
| Rapeseed oil | 29.11 |
| Saponin | 13.09 |
| Fructose | 26.17 |
| H₂O | 26.17 |

Firstly, an oil phase solution was prepared. 2.5 g Phytosterol (β-sitosterol), 24 g β-ethylcholesterol (40% pure, including campesterol and dihydrobrassicasterol, purchased from Sigma Co.) and 2.5 g propylene glycol monostearate 90 (abbreviated as PGMS 90, with HLB value of 3.4) were dissolved in 26.7 g of an edible Rapeseed oil (Canola oil from Taiwan Sugar Co.). Next, a water phase solution was prepared. 12 g Tea saponin (Taiwan Sansui Organic Tech. Co., Ltd.) and 24 g fructose (Fong Leng Fructose from Taiwan Sugar Co.) were homogenously dissolved in 24 g of deionized water. Finally, the oil phase solution and the water phase solution were vigorously mixed in a magnet mixer at 80° C. in order to achieve a uniform and stable mixing. After 7-day of observation, the phytosterol emulsion was found to be still stable.

The above procedures in preparing a water phase solution was repeated wherein Tea saponin was replaced by Sapindaceae (Taiwan Sansui Organic Tech. Co., Ltd.), Yucca extract, and Quillaia extract (Garuda Int., USA), respectively. The experimental results indicate that the four saponins used are all able to render phytosterol achieving a stable emulsification state.

### EXAMPLE 2

In Example 1, a synthetic surfactant (propylene glycol monostearate 90) and grease were used to dissolve phytosterol. In Example 2, only heated isopropanol was used to dissolve phytosterol.

5 g phytosterol was dissolved in 100 mL isopropanol at 60° C. Next, 5 g of various saponins (Tea saponin, Sapindaceae, Yucca extract, and Quillaia extract) were separately dissolved in 100 ml water at 60° C. to replace the water phase solution in Example 1. Similarly, the oil phase solution and the water phase solution were vigorously mixed in a magnet mixer at 60° C. in order to achieve a uniform and stable mixing effect. Isopropanol was substantially all removed from the resulting emulsions in vacuo. The dispersion stability of phytosterol in the remaining water phases (oil-in-water emulsions of phytosterol) was observed.

After 7-day of observation, the phytosterol emulsions were found still stable. The phytosterol emulsions were frozen dried to obtain various miniaturized phytosterol powder products containing different saponins. Next, the dried powder products were added with water again to observe the dispersion property of the dried powder products in water. The results indicate that the powder product prepared from phytosterol and tea saponin and or the powder product prepared from phytosterol and Quillaia extract have the best water dispersion performance.

1. A method for emulsifying phytosterol, which comprises the following steps:
a) dissolving phytosterol in an oil phase;
b) dissolving a saponin in a water phase; and
c) mixing said oil phase solution from step a) and said water phase solution from step b) to form an emulsion.

2. The method as claimed in claim 1, which further comprises:
d) removing a major portion of said oil phase from said emulsion by evaporation under a reduced pressure; and
e) drying the resulting oil-in-water emulsion from step d) to obtain a phytosterol powder product containing said saponin and phytosterol, which is capable of being re-dispersed in a water phase.

3. The method as claimed in claim 2, wherein said oil phase in step a) comprises an organic solvent with a boiling point lower than water, and said step d) comprises distilling said emulsion from step c).

4. The method as claimed in claim 3, wherein said oil phase in step a) comprises isopropanol.

5. The method as claimed in claim 1, wherein said oil phase in step a) comprises a vegetable oil.

6. The method as claimed in claim 1, wherein said oil phase in step a) comprises an animal oil.

7. The method as claimed in claim 1, wherein said saponin is a natural saponin extracted from a plant.

8. The method as claimed in claim 7, wherein said plant is selected from the group consisting of Camellia Oleifera plant, Sapindus plant, Quillaia plant, and Yucca plant.

9. A phytosterol emulsion prepared according to the method claimed in claim 1.

10. A phytosterol powder product containing saponin and phytosterol and prepared according to the method claimed in claim 2.

11. A phytosterol powder product containing saponin and phytosterol and prepared according to the method claimed in claim 3.

12. A phytosterol powder product containing saponin and phytosterol and prepared according to the method claimed in claim 4.
